(19) United States
(12) Patent Application Publication
(10) Pub. No.: US 2020/0205880 A1
(43) Pub. Date: Jul. 2, 2020

(54) ELECTRODE FOR HIGH FREQUENCY MEDICAL DEVICE AND HIGH FREQUENCY MEDICAL DEVICE

(71) Applicant: OLYMPUS CORPORATION, Tokyo (JP)

(72) Inventors: Hiroaki KASAI, Tokyo (JP); Yu MURANO, Hirosaki-shi (JP); Yoshiyuki OGAWA, Tokyo (JP); Takuya FUJIHARA, Tokyo (JP)

(73) Assignee: OLYMPUS CORPORATION, Tokyo (JP)

(21) Appl. No.: 16/811,608

(22) Filed: Mar. 6, 2020

Related U.S. Application Data
(63) Continuation of application No. PCT/JP2018/037968, filed on Oct. 11, 2018.

(30) Foreign Application Priority Data
Oct. 20, 2017 (JP) 2017-203665

(51) Int. Cl. A61B 18/14

(52) U.S. Cl. CPC A61B 18/14; A61B 2018/00601

(57) ABSTRACT

An electrode for high frequency medical device includes: a base; and a coating layer which is laminated onto the base, includes at least either one of a fluororesin and a silicon compound, has a volume resistivity of 1.0×10⁰ to 1.0×10¹³Ω·×cm, and has a thickness of 1 to 30 μm. The electrode is configured to denature and dissect a living tissue by a Joule heating due to a high frequency current and an arc discharge.

### CROSS REFERENCE TO RELATED APPLICATIONS

This application is a continuation application based on a PCT Patent Application No. PCT/JP2018/037968, filed on Oct. 11, 2018, whose priority is claimed on Japanese Patent Application No. 2017-203665, filed Oct. 20, 2017. The contents of both the PCT Application and the Japanese Application are incorporated herein by reference.

### BACKGROUND

### Technical Field

The present invention relates to an electrode for high frequency medical device and a high frequency medical device.

### Background Art

As a high frequency medical device, a device for applying a high frequency voltage to a living tissue is known. Such high frequency medical device is used for performing treatment on the living tissue by applying a high frequency voltage to the living tissue. For example, the high frequency medical device can incise, coagulate, or cauterize the living tissue.

In an electrode for the high frequency medical device, coating of a non-adhesive substance on the electrode surface may be used in order to prevent the living tissue from adhering to the electrode surface.

However, since the non-adhesive substance has electrical insulation, there is a problem that the high frequency characteristics as an electrode are lowered and the incision property is deteriorated.

For this reason, Published Japanese Translation No. 2001-518344 of the PCT International Publication (hereinafter referred to as Patent Document 1) proposes to use an electrosurgical electrode member including a conductive electrode having an edge part with a width of 0.2 mm or less, for the purpose of obtaining good incision properties even when the electrode surface is covered with a non-adhesive substance.

In the electrosurgical electrode member of Patent Document 1, since the conductive electrode has an edge part with a width of 0.2 mm or less, the current density in the vicinity of the edge part is increased. For this reason, according to the electrosurgical electrode member of Patent Document 1, the incision can be made even when the conductive electrode is covered with a non-adhesive substance that is an insulator.

However, in the electrosurgical electrode member of Patent Document 1, high frequency energy concentrates on the non-adhesive substance in the vicinity of the edge part, and therefore, the non-adhesive substance is easily deteriorated and the non-adhesive substance is peeled off from the electrode surface. For this reason, even when the incision property improves, the useful life of the electrosurgical electrode member will become short.

In view of the above, the present invention provides an electrode for high frequency medical device and a high frequency medical device which can maintain favorable treatment performance for a long period of time.

### SUMMARY

An electrode for high frequency medical device includes: a base; and a coating layer laminated onto the base, including at least either one of a fluororesin and a silicon compound, having a volume resistivity of 1.0×10⁰ to 1.0×10¹³Ω·cm, and having a thickness of 1 to 30 μm. The electrode is configured to denature and dissect a living tissue by a Joule heating caused by a high frequency current and an arc discharge.

In the electrode for high frequency medical device, the electrode may have a curved surface having a radius of curvature larger than 0.1 mm.

In the electrode for high frequency medical device, the coating layer may contain carbon particles.

In the electrode for high frequency medical device, the coating layer may have a thickness of 5 μm or more and 30 μm or less.

A high frequency medical device comprising an electrode, wherein the electrode includes: a base; and a coating layer laminated onto the base, including at least either one of a fluororesin and a silicon compound, having a volume resistivity of 1.0×10⁰ to 1.0×10¹³Ω ·cm, and hving a thickness of 1 to 30 μm, and the electrode being configured to denature and dissect a living tissue by a Joule heating caused by a high frequency current and an arc discharge.

### BRIEF DESCRIPTION OF THE DRAWINGS

FIG. 1 is a schematic configuration diagram showing an example of a high frequency medical device according to an embodiment of the present invention.

FIG. 2 is a cross-sectional view taken along line A-A in FIG. 1.

FIG. 3 is a schematic cross-sectional view of an electrode for high frequency medical device according to an embodiment of the present invention.

### DETAILED DESCRIPTION OF THE PREFERRED EMBODIMENTS

An electrode for high frequency medical device and a high frequency medical device of an embodiment of the present invention will be described with reference to accompanying drawings.

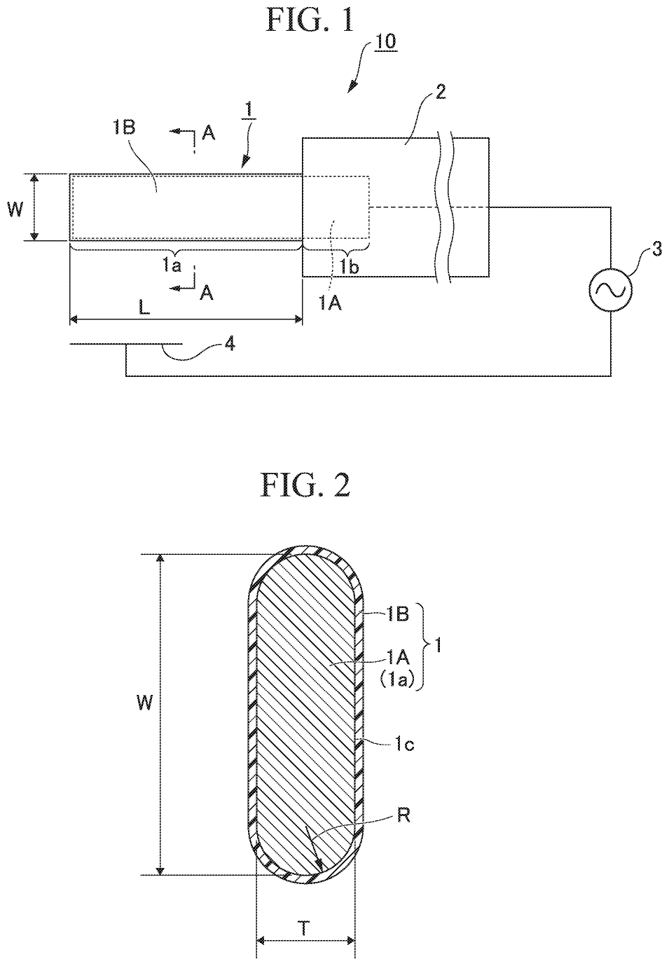

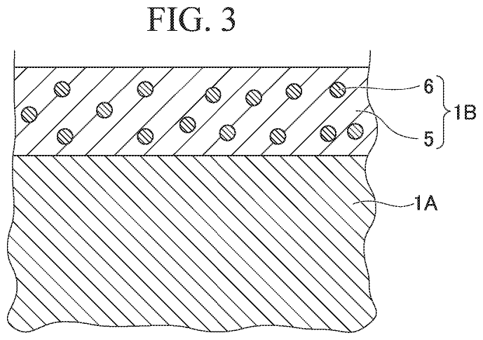

FIG. 1 is a schematic configuration diagram showing an example of a high frequency medical device according to an embodiment of the present invention. FIG. 2 is a cross-sectional view taken along the line A-A in FIG. 1. FIG. 3 is a schematic cross-sectional view of an electrode for high frequency medical device according to an embodiment of the present invention.

A high frequency knife 10 shown in FIG. 1 is an example of a high frequency medical device of this embodiment. The high frequency knife 10 is a medical treatment instrument that applies a high frequency voltage to incise and excise a living tissue, and coagulate (hemostasis) or cauterize the living tissue.

The high frequency knife 10 includes a rod-shaped grasping part 2 for an operator to hold by hand, and an electrode part 1 (electrode for high frequency medical device) protruding from the distal end of the grasping part 2. The grasping part 2 and the electrode part 1 are electrically insulated from each other.

The electrode part 1 is in contact with a living tissue that is an object to be treated and applies a high frequency voltage to the living tissue.

The electrode part 1 includes an electrode body 1A (base) and a coating layer 1B.

The electrode body 1A is made of a metal material having good conductivity. When the material of the electrode body 1A is a metal material having excellent workability, a complicated electrode shape can be easily formed.

Examples of metal materials suitable for the electrode body 1A include stainless steel, aluminum, aluminum alloy, titanium, and titanium alloy.

In the electrode body 1A, the shape of the fixed end 1b covered with the grasping part 2 is an appropriate shape that can be easily fixed to the grasping part 2.

As the shape of the protruding part 1a protruding from the grasping part 2 in the electrode body 1A, an appropriate shape according to the treatment application of the electrode part 1 is used. For example, the shape of the protruding part 1a may be a plate shape, a round bar shape, a square bar shape, a disk shape, or a hook shape.

However, as will be described later, in the case of the electrode body 1A, good treatment performance can be obtained without providing a sharp edge part in a portion that comes into contact with the living tissue via the coating layer 1B described later during use. For this reason, even when it aims at incision for example, it is not necessary to provide a sharp edge part in electrode body 1A. Here, the “sharp edge part” means a rounded edge part having a radius of curvature of 0.1 mm or less in a cross section perpendicular to the edge, or a substantially V-shaped edge part having a width of 0.2 mm or less at the distal end of the edge in a cross section perpendicular to the edge.

Similarly, it is not necessary that a sharp needle-like part is provided in the electrode body 1A. Here, the “sharp needle-like part” means a needle-like part whose radius of curvature of the curved surface at the distal end is 0.1 mm or less, or a needle-like part whose distal end surface has a diameter of 0.2 mm or less.

As an example, the shape of the protruding part 1a of the electrode body 1A shown in FIGS. 1 and 2 is a rectangular plate. The length×width×thickness of the protruding part 1a is represented byL×W×T (where T<W<L). However, as shown in FIG. 2, both side surfaces in the short width direction (the vertical direction in the figure) of the protruding part 1a are curved surfaces rounded to a curvature radius R (where R=T/2).

In the electrode body 1A, the thickness T is more preferably less than 0.2 mm and the radius of curvature R is more than 0.1 mm.

As shown in FIG. 1, the electrode body 1A is electrically connected to the high frequency power source 3 by wiring connected to the fixed end 1b in the grasping part 2. The high frequency power source 3 is electrically connected to a counter electrode plate 4 to be attached to the object to be treated.

As shown in FIGS. 1 and 2, the coating layer 1B is formed of a thin film that is laminated on the electrode body surface 1c of the electrode body 1A and covers at least the entire protruding part 1a.

The coating layer 1B is configured to include at least one of a fluororesin and a silicon compound for the purpose of decreasing adhesion of the living tissue.

Furthermore, the coating layer 1B is configured such that the volume resistivity is 1.0×10⁰Ω·cm to 1.0×10¹³Ω·cm, and the layer thickness is 1 μm to 30 μm.

The layer thickness of the coating layer 1B is more preferably 5 μm or more and 30 μm or less.

For example, as the fluororesin contained in the coating layer 1B, one or more materials selected from the group consisting of PTFE (polytetrafluoroethylene), PFA (tetrafluoroethylene/perfluoroalkyl vinyl ether copolymer), FEP (tetrafluoroethylene/hexafluoropropylene copolymer), ETFE (tetrafluoroethylene/ethylene copolymer), and PCTFE (polychlorotrifluoroethylene) may be used. For example, as the fluororesin contained in the coating layer 1B, KH-100 (trade name, manufactured by Kawamata Laboratories) may be used.

For example, as the silicon compound contained in the coating layer 1B, one or more materials selected from the group consisting of silicone resin, silicone rubber, and silica, silicone resin or silicone rubber having a modifying methyl group on the surface may be used.

For example, as the silicon compound contained in the coating layer 1B, silicone resin KR-251 (trade name, manufactured by Shin-Etsu Chemical Co., Ltd.) may be used.

When the above-described volume resistivity is realized by at least one of the fluororesin and the silicon compound, the coating layer 1B may be configured by only at least one of the fluororesin and the silicon compound.

However, in order to achieve the above-described volume resistivity, the coating layer 1B may include a conductive additive that adjusts the electrical resistance.

For example, as schematically shown in FIG. 3, the coating layer 1B may include a base material 5 containing at least one of a fluororesin and a silicon compound, and a conductive filler 6 (conductive additive) dispersed in the base material 5.

The content of the conductive filler 6 is adjusted according to the electric resistance of the base material 5 so that the above-described volume resistivity is obtained in the coating layer 1B.

For example, the coating layer 1B may be formed of a mixture (for example, the above-described KH-100) provided by previously mixing the uncured material serving as the base material 5 and the conductive filler 6. For example, the coating layer 1B may be formed of a material obtained by further adding another conductive filler 6 to a mixture provided by previously mixing an uncured material serving as the base material 5 and the conductive filler 6.

For example, the conductive filler 6 may include a carbon filler, a metal filler, and a metal film filler.

The carbon filler may be in the form of particles (powder) like conductive carbon black, in the form of fiber like PAN-type or pitch-type carbon fiber or carbon nanotube (CNT), or in the form of flake (plate) like graphite or graphene plate. Metal fillers may in the form of powders such as Au (gold), Ag (silver), Ni (nickel), Cu (copper), Zn (zinc), Al (aluminum), or SUS (stainless steel), in the form of flake such as Ag, Ni, Cu, Zn or Al, or in the form of fiber such as Cu or SUS.

The metal film filler may be constituted by coating a base filler such as particulate (powder) or fibrous mica, glass beads, glass fiber, calcium carbonate, titanium oxide, or the like with Ni or Al.

The metal filler has an advantage of being able to reduce the addition amount because they are more conductive than the carbon filler. The metal film filler has an advantage that it can be toned by selecting the color of the base filler.

In particular, when flaky graphite is used as the carbon filler, conductivity is improved due to the development of crystallinity. Further, when fibrous carbon is used as the carbon filler, it is fibrous and can be entangled with each other to improve strength.

The electrode part 1 described above may be manufactured as follows, for example.

An appropriate metal material is processed to produce the electrode body 1A. Examples of the method for manufacturing the electrode body 1A include press processing, cutting processing, and molding processing.

Thereafter, the coating layer 1B is formed on the electrode body surface 1c of the electrode body 1A.

The coating layer 1B may be formed by application, for example. In this case, the conductive filler 6 is first mixed with the paint containing the components of the base material 5. The addition amount of the conductive filler 6 is an amount determined so as to obtain a volume resistivity required when the paint is cured. The necessary addition amount of the conductive filler 6 can be determined in advance by experiments or the like.

In this manner, the coating material for forming the coating layer 1B is prepared.

Thereafter, this coating material is applied to the electrode body surface 1c by an appropriate coating means. The application means is not particularly limited.

Examples of the application means include spray coating, dip coating, spin coating, screen printing, ink jet method, flexographic printing, gravure printing, pad printing, hot stamping, and the like. Spray coating and dip coating are particularly suitable as application means for forming the coating layer 1B on a high frequency medical device because they can be easily applied even when the shape of the object to be coated is complicated.

For example, the paint layer formed on the coating layer 1B is dried by being heated or the like. Thereby, the coating layer 1B is formed.

Thus, the electrode part 1 is manufactured.

Next, the operation of the high frequency knife 10 and the electrode part 1 having such a configuration will be described. First, operation and usage method of the high frequency knife 10 and the electrode part 1 will be described.

As shown in FIG. 1, for example, treatment using the high frequency knife 10 is performed in a state where a counter electrode 4 is attached to a patient (not shown) and a high frequency voltage is applied to the electrode part 1 by the high frequency power source 3. The surgeon brings the electrode part 1 into contact with the object to be treated such as a patient's part to be treated while the high frequency voltage is applied to the electrode part 1. For example, in order to perform an incision of the living tissue, either one of both end portions in the width direction of the electrode part 1 whose distal end is rounded may be brought into contact with the living tissue. For example, in order to coagulate and cauterize the living tissue, any one of the flat portions formed in the thickness direction of the electrode part 1 may be brought into contact with the living tissue.

When a high frequency voltage is applied between the electrode part 1 and the counter electrode plate 4, a high frequency current is generated between the electrode part 1 and the living tissue via the coating layer 1B. A Joule heating is generated when the high frequency current flows through the living tissue. Thereby, the moisture of the living tissue in the object to be treated evaporates rapidly, and the living tissue is dissected by the pressing force from the electrode part 1. For this reason, the incision and excision of the living tissue can be performed by moving the electrode part 1 relative to the living tissue.

When a high frequency current is applied in a state where the electrode part 1 is pressed against the object to be treated, the moisture of the living tissue in the object to be treated evaporates rapidly, and the living tissue is coagulated in the vicinity of the electrode part 1. For this reason, when the electrode part 1 is pressed against the object to be treated, hemostasis or cauterization of the living tissue can be performed.

When the necessary treatment is completed, the surgeon moves the electrode part 1 away from the object to be treated. At this time, since the living tissue is hardly attached to the coating layer 1B in contact with the living tissue by the base material 5, the living tissue is easily peeled off.

Next, the effect of the coating layer 1B in the electrode part 1 will be described in detail compared with related art.

For example, the non-adhesive substance used for covering the electrode in Patent Document 1 described above is an insulator, and, the volume resistivity of the non-adhesive substance used for coating such an electrode is about 1.0×10¹⁴Ω·cm to 1.0×10¹⁵Ω·cm or larger than this range.

The high frequency characteristics of the electrode covered with such an insulator are degraded. For this reason, treatment performance such as incision is lowered in the electrode covered with the insulator as compared with the metal electrode not having the coating. In order to compensate for such a decrease in treatment performance, it is considered to increase the current density when a high frequency voltage is applied. For example, when the edge part is provided in the electrode body as in the art described in Patent Document 1, the electric field distribution is concentrated on the edge part, thereby increasing the current density in the vicinity of the edge part.

However, as the current density increases, the coating damage also increases, so that the coating itself deteriorates or the coating peels off from the electrode surface. Thereby, the useful life of the electrode part may fall.

As a result of intensive investigations, the present inventors have found that sparks caused by electric discharges play an important role to improve the treatment performance of the coated electrode, and this finding paved a way to the present invention.

According to the study of the present inventors, in the living tissue in contact with the conductive electrode surface, moisture evaporation and protein denaturation occur due to a Joule heating, whereby an insulating layer derived from the living tissue is formed on the electrode surface. When a high frequency voltage is further applied in this state, a minute arc discharge is generated from the electrode surface through the insulating layer. It is considered that treatment such as incision proceeds smoothly by the energy of this arc discharge promoting the degeneration and dissection of the living tissue.

The present inventors came to think that a favorable treatment performance could be obtained without damaging the coating, by generating a minute arc discharge in a wide area of the electrode surface, not concentrating the current density on the edge part as in the art described in Patent Document 1.

As a result of repeated experiments, the inventors has found that such a small are discharge is likely to occur when the coating layer 1B has a volume resistivity of 1.0×10⁰Ω·cm to 1.0×10¹³Ω·cm and a layer thickness of 1 μm to 30 μm.

According to the coating layer 1B of the present embodiment, when the volume resistivity and the layer thickness satisfy the above-described ranges, the electrical insulation is relaxed and the withstand voltage is reduced. For this reason, even when the electric field density is low, an arc discharge from the electrode part 1 is likely to occur. Such are discharge easily occurs even when a sharp edge part is not formed on the electrode body surface 1c, and therefore occurs in a wide area on the electrode body surface 1c.

As a result, the living tissue in contact with the coating layer 1B receives discharge energy concentrated on the discharge path of the arc discharge along with a Joule heating generated due to the high frequency current. In particular, since the discharge path of the are discharge locates in a small area, large heat is generated locally due to the concentration of discharge energy. For this reason, the living tissue is microscopically denatured and dissected over a wide area.

Thus, according to the electrode part 1 of the present embodiment, since the modification and dissection of the living tissue progress over the whole contact area of the electrode part 1 and the living tissue, treatment can be performed smoothly. For example, at the time of incision, the sharpness of the incision is improved and the incision can be easily and rapidly advanced.

On the other hand, since the load received by the coating layer 1B due to the discharge energy is dispersed throughout the contact area with the living tissue, deterioration of the coating layer 1B is suppressed. That is, the deterioration of the adhesion preventing performance of the living tissue is suppressed by reducing the damage of the molecular structure of the base material 5. Furthermore, since damage at the interface between the base material 5 and the electrode body surface 1c is reduced, peeling of the coating layer 1B is suppressed.

Thus, the electrode part 1 and the high frequency knife 10 of the present embodiment can maintain favorable treatment performance for a long time. As a result, the useful life of the high frequency knife 10 and the electrode part 1 is improved.

In the description of the above embodiment, the high frequency medical device including the electrode for the high frequency medical device is described as an example of a high frequency knife, but the high frequency medical device is not limited to the high frequency knife. Examples of other high frequency medical devices in which the electrode for high frequency medical device of the present invention can be suitably used include treatment tools such as a high frequency scissor knife, an electric knife, and a snare.

In the description of the above embodiment, an example in which the electrode body 1A has a rectangular plate shape with a constant thickness and roundness is formed at the end in the width direction has been described. However, the plate shape suitable for the electrode body 1A is not limited to a plate shape having a constant thickness. For example, the electrode body 1A may have a plate shape in which the plate thickness gradually decreases toward the outer edge part. However, it is more preferable that the shape of the distal end of the outer edge part is not the sharp edge part described above.

### EXAMPLE

Next, Examples 1 to 4 of electrodes for high frequency medical devices corresponding to the above-described embodiment will be described together with Comparative Examples 1 to 5. Table 1 below shows the configurations of the electrode parts and evaluation results of the examples and comparative examples.

TABLE 1

| | | COATING LAYER | | | | |
|---|---|---|---|---|---|---|
| | | | CONDUCTIVE FILLER | VOLUME | FILM | |
| | BASE | | CONTENT RATE | RESISTIVITY | THICKNESS | DURABILITY |
| | MATERIAL | BASE MATERIAL | (mass %) | (Ω · cm) | (μm) | EVALUATION |
| EXAMPLE 1 | SUS304 | FLUORINE RESIN | 0.01 | 1.0 × 10⁰ | 30 | ◯ |
| EXAMPLE 2 | SUS304 | FLUORINE RESIN | 0.01 | 1.0 × 10⁰ | 1 | ◯ |
| EXAMPLE 3 | SUS304 | SILICONE RESIN | 8 | 1.0 × 10¹³ | 30 | ◯ |
| EXAMPLE 4 | SUS304 | SILICONE RESIN | 8 | 1.0 × 10¹³ | 1 | ◯ |
| COMPARATIVE | SUS304 | FLUORINE RESIN | 0.01 | 1.0 × 10⁰ | 40 | X |
| EXAMPLE 1 | | | | | | |
| COMPARATIVE | SUS304 | FLUORINE RESIN | 0.01 | 1.0 × 10⁰ | 0.5 | X |
| EXAMPLE 2 | | | | | | |
| COMPARATIVE | SUS304 | FLUORINE RESIN | 0.1 | 1.0 × 10⁻¹ | 20 | X |
| EXAMPLE 3 | | | | | | |
| COMPARATIVE | SUS304 | SILICONE RESIN | 0 | 1.0 × 10¹⁵ | 20 | X |
| EXAMPLE 4 | | | | | | |
| COMPARATIVE | SUS304 | — | — | — | — | X |
| EXAMPLE 5 | | | | | | |

### Example 1

Example 1 is an example corresponding to the electrode part 1 of the above-described embodiment.

As shown in Table 1, stainless steel SUS304 was used as the material of the electrode body 1A as the base. The protruding part 1a of the electrode body 1A was formed in a rectangular plate shape as shown in FIGS. 1 and 2. The cross-sectional shape of the electrode body 1A was T=0.5 mm and R=0.25 mm. Hereinafter, the shape of the electrode body 1A of Example 1 is referred to as #1.

A fluororesin was used as the base material 5 of the coating layer 1B (reference numerals are omitted in Table 1, and the names of other members are the same). Specifically, as the base material 5, a cured product of KH-100 (trade name, manufactured by Kawamata Laboratories Co., Ltd.), which is a fluororesin paint, was used. KH-100 contains carbon black that constitutes a part of the conductive filler 6.

In addition to carbon black in KH-100, Denka Black (trade name, manufactured by Denka Co., Ltd.) was further added as the conductive filler 6 of the coating layer 1B. Denka Black is a kind of acetylene black of carbon black. The content of Denka Black in the coating layer 1B was set to 0.01 mass % so that the volume resistivity of the coating layer 1B was 1.0×10⁰Ω·cm.

The layer thickness of the coating layer 1B was 30 μm.

The electrode part 1 of Example 1 was manufactured as follows.

The coating material to be the base material 5 and the conductive filler 6 were mixed after being weighed so that the content of the conductive filler 6 was the above-described value at the time of curing. Thereby, the coating material which forms the coating layer 1B was manufactured.

This coating material was spray-coated on the electrode body surface 1c of the electrode body 1A after the electrode body 1A was manufactured. Thereafter, the coating film was cured by heating at 380° C. for 1 hour. In this way, the coating layer 1B was formed on the electrode body 1A. Thereby, the electrode part 1 of Example 1 was manufactured.

The electrode part 1 was attached with the grasping part 2 after the wiring was connected. The wiring of the electrode part 1 was electrically connected to the high frequency power source 3 to which the counter electrode plate 4 was connected. Thus, the high frequency knife 10 of Example 1 was manufactured.

### Example 2

Example 2 was configured in the same manner as Example 1 except that the layer thickness of the coating layer 1B was 1 μm. The electrode part 1 and the high frequency knife 10 of Example 2 were manufactured in the same manner as Example 1 except that the thickness of the coating layer 1B was 1 μm.

### Examples 3 and 4

Example 3 differs from Example 1 in the material of the base material 5 and the content of the conductive filler 6 added to the base material 5.

As the base material 5, a silicone resin containing a silicon compound was used. Specifically, a cured product of silicone resin KR-251 (trade name, manufactured by Shin-Etsu Chemical Co., Ltd.) was used as the base material 5.

Since KR-251 does not contain a material to be the conductive filler 6, the same conductive filler 6 as that added to KH-100 in Example 1 was added.

The content of the conductive filler 6 in the coating layer 1B was 8 mass % so that the volume resistivity of the coating layer 1B was 1.0×10¹³Ω·cm.

The electrode part 1 and the high frequency knife 10 of Example 3 were manufactured in the same manner as Example 1 except that the material of the base material 5 and the addition amount of the conductive filler 6 were different.

Example 4 was configured in the same manner as Example 3 except that the thickness of the coating layer 1B was 1 μm. The electrode part 1 and the high frequency knife 10 of Example 4 were manufactured in the same manner as Example 3 except that the thickness of the coating layer 1B was 1 μm.

### Comparative Examples 1 to 5

Comparative Examples 1 and 2 were configured in the same manner as in Example 1 except that the coating layer thickness was 40 μm and 0.5 μm, respectively.

Comparative Example 3 is an example in which the material is the same as in Example 1 and the volume resistivity and the layer thickness are changed. In Comparative Example 3, the volume resistivity of the coating layer was set to 1.0×10-1 Ω·cm by setting the content of the conductive filler 6 to 0.1 mass %. In Comparative Example 3, the thickness of the coating layer was 20 μm.

Comparative Example 4 is an example in which the coating layer is composed only of the base material 5 used in Example 2 (the content of the conductive filler 6 is 0 mass %). The coating layer of Comparative Example 4 was formed by forming the base material 5 used in Example 2 on the electrode body surface 1c so as to have a layer thickness of 20 μm. As a result, the volume resistivity of the coating layer of Comparative Example 4 was 1.0×10¹⁵Ω·cm.

In Comparative Example 5, the electrode part was composed of only the electrode body 1A.

### (Evaluation Methods)

Evaluation of the durability of the living tissue in the electrode parts of Examples 1 to 4 and Comparative Examples 1 to 5 was performed.

The evaluation of the durability was performed by repeating the simulated organ tissue incision test with the high frequency knife of each Example and each Comparative Example.

The object to be incised was a pig gastric mucosa. The incision conditions were all in the coagulation/incision mixed mode and the output of 50 W.

### (Evaluation Results)

Table 1 describes the evaluation results of the evaluation of the durability.

In Examples 1 to 4, a spark was generated and a good incision was made. In Examples 1 to 4, a good incision could be made even after the simulated organ tissue incision test was repeated 100 times or more.

Comparative Examples 1 to 5 all had a problem in incision performance, so they were evaluated as “bad” (no good, described as “x” in Table 1).

Specifically, in Comparative Examples 1 and 4, incision was impossible from the first time, so they were evaluated as “bad”.

In Comparative Examples 1 and 4, no spark occurred.

In Comparative Example 1, it is considered that the electrical resistance of the coating layer was too high because the coating layer was too thick.

Since Comparative Example 4 does not include a conductive filler, it is considered that the electrical resistance of the coating layer is too high because the volume resistivity itself is too large.

As described above, in Comparative Examples 1 and 4, it was considered that the incision was impossible because the electric resistance of the coating layer was too high to generate a spark.

In Comparative Examples 2 and 3, sparks occurred and the incision could be performed 100 times or more. However, the incisibility was inferior to each of the Examples, and thus they were evaluated as “bad”.

Specifically, there was a delay of about 0.5 seconds from when the high frequency voltage was applied to when the spark was generated and the incision could be started. The high frequency knives of Comparative Examples 2 and 3 had such incision characteristics. However, if this delay can be eliminated, it is considered that the operator can faithfully reflect the timing at which the operator wants to incise, and a smoother operation can be realized.

In Comparative Example 2, the layer thickness of the coating layer is too thin, and in Comparative Example 3, the volume resistivity is too low, so it is considered that the electric resistance becomes too low. For this reason, it is considered that an arc discharge did not occur until an insulating layer due to denaturation of living tissue was formed to some extent on the surface of the electrode part. For this reason, it is considered that it took a considerable time before the incision could be started.

Comparative Example 5 was evaluated as “bad” because the incision was impossible at the third time. Furthermore, the first and second incisions were much inferior to those in each Example. Specifically, since the electrode part of Comparative Example 5 did not include a coating layer on the surface, the living tissue adhered to the surface at the time of the third incision, and the incision was impossible. Even at the first and second incisions, as in Comparative Examples 2 and 3, it took time until the incision could be started, so the incisibility was poor.

Thus, in each Example, since the coating layer 1B was appropriately formed, good incisability as the electrode part 1 could be maintained for a long time.

On the other hand, since each Comparative Example did not have an appropriate coating layer, good incisability could not be obtained, or good incisability could not be maintained for a long time.

As described above, although preferable embodiment of this invention was described with each example, this invention is not limited to these embodiment and each example. Additions, omissions, substitutions, and other modifications can be made without departing from the spirit of the present invention.

Further, the present invention is not limited by the above description and is limited only by the appended claims.

The present invention can be widely applied to an electrode for high frequency medical device and a high frequency medical device, and good treatment performance can be maintained for a long time.

What is claimed is:

1. An electrode for high frequency medical device comprising:
a base; and
a coating layer which is laminated onto the base, including at least either one of a fluororesin and a silicon compound, having a volume resistivity of 1.0×10⁰ to 1.0×10¹³Ω·cm, and having a thickness of 1 to 30 μm,
wherein the electrode is configured to denature and dissect a living tissue by a Joule heating due to a high frequency current and an arc discharge.

2. The electrode for high frequency medical device according to claim 1, wherein the electrode has a curved surface having a radius of curvature larger than 0.1 mm.

3. The electrode for high frequency medical device according to claim 1, wherein the coating layer contains carbon particles.

4. The electrode for high frequency medical device according to claim 1, wherein the coating layer has a thickness of 5 μm or more and 30 μm or less.

5. A high frequency medical device comprising an electrode, wherein the electrode includes:
a base; and
a coating layer which is laminated onto the base, including at least either one of a fluororesin and a silicon compound, having a volume resistivity of 1.0×10⁰ to 1.0×10¹³Ω·cm, and has a thickness of 1 to 30 μm, and
the electrode being configured to denature and dissect a living tissue by a Joule heating due to a high frequency current and an arc discharge.
